SEQUENCE LISTING (24 sequences)

SEQ ID NO: 1 - protein, 120 aa, Homo sapiens
QVQLQESGPGLVKPSETLSLTCTVSGGSISSYYWSWIRQPPGKGLEWIGYFFYSGYTNYNPSLKSRVTISVDTSKNQFSLKLSSVTAADTAVYYCACITGTTKGGMDVWGQGTTVTVSSA

SEQ ID NO: 2 - protein, 112 aa, Homo sapiens
QSVLTQPPSVSGAPGQRVTISCTGSSSNIGAGYDVHWYQQLPGTAPKLLIYGNNNRPSGVPDRFSGSKSGTSASLAITGLQADDEADYYCQSFDSSLSGSVFGGGTKLTVLG

SEQ ID NO: 3 - protein, 120 aa, Homo sapiens
QVQLQESGPGLVKPSETLSLTCTVSGGSISSYYWSWIRQPPGRGLEWIGYFFYSGYTNYNPSLKSRVTMSVDTSKNQFSLKLSSVTAADTAVYYCACITGTTKGGMDVWGQGATVTVSSA

SEQ ID NO: 4 - protein, 112 aa, Homo sapiens
QSVLTQAPSVSGAPGQRVTISCTGRSSNIGAGYDVHWYQQFPGTAPKLLIYGNSNRPSGVPDRFSGSKSGTSASLAITGLQAEDEADYYCQSYDSSLSGSVFGGGTKLTVLG

SEQ ID NO: 5 - protein, 121 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSA

SEQ ID NO: 6 - protein, 112 aa, Homo sapiens
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLG

SEQ ID NO: 7 - protein, 5 aa, Homo sapiens
SYYWS

SEQ ID NO: 8 - protein, 16 aa, Homo sapiens
YFFYSGYTNYNPSLKS

SEQ ID NO: 9 - protein, 11 aa, Homo sapiens
ITGTTKGGMDV

SEQ ID NO: 10 - protein, 14 aa, Homo sapiens
TGSSSNIGAGYDVH

SEQ ID NO: 11 - protein, 7 aa, Homo sapiens
GNNNRPS

SEQ ID NO: 12 - protein, 11 aa, Homo sapiens
QSFDSSLSGSV

SEQ ID NO: 13 - protein, 5 aa, Homo sapiens
SYYWS

SEQ ID NO: 14 - protein, 16 aa, Homo sapiens
YFFYSGYTNYNPSLKS

SEQ ID NO: 15 - protein, 11 aa, Homo sapiens
ITGTTKGGMDV

SEQ ID NO: 16 - protein, 14 aa, Homo sapiens
TGRSSNIGAGYDVH

SEQ ID NO: 17 - protein, 7 aa, Homo sapiens
GNSNRPS

SEQ ID NO: 18 - protein, 11 aa, Homo sapiens
QSYDSSLSGSV

SEQ ID NO: 19 - protein, 5 aa, Homo sapiens
SYDIN

SEQ ID NO: 20 - protein, 17 aa, Homo sapiens
WMNPNSGNTGYAQKFQG

SEQ ID NO: 21 - protein, 11 aa, Homo sapiens
DPYYYYYGMDV

SEQ ID NO: 22 - protein, 13 aa, Homo sapiens
SGSSSNIENNHVS

SEQ ID NO: 23 - protein, 7 aa, Homo sapiens
DNNKRPS

SEQ ID NO: 24 - protein, 12 aa, Homo sapiens
ETWDTSLSAGRV

(19) United States
(12) Patent Application Publication
(10) Pub. No.: US 2015/0361128 A1
(43) Pub. Date: Dec. 17, 2015

(54) METHODS OF USING ION EXCHANGE CHROMATOGRAPHY TO CONTROL LEVELS OF HIGH MANNOSE GLYCOFORMS

(71) Applicant: MEDIMMUNE, LLC, Gaithersburg, MD (US)

(72) Inventors: Saravanamoorthy RAJENDRAN, Gaithersburg, MD (US); Jihong WANG, Gaithersburg, MD (US)

(21) Appl. No.: 14/653,531

(22) PCT Filed: Dec. 18, 2013
(86) PCT No.: PCT/US13/76002

§ 371 Date: Jun. 18, 2015

Related U.S. Application Data
(60) Provisional application No. 61/740,238, filed on Dec. 20, 2012.

(51) Int. Cl. C07K 1/18; C07K 16/00

(52) U.S. Cl. CPC C07K 1/18; C07K 16/00; C07K 2317/565

(57) ABSTRACT

Provided are methods of using ion exchange based chromatography to control the levels of high mannose glycoforms in a glycoprotein sample, such as an antibody sample. The high mannose glycoforms can be removed from the sample or isolated and enriched. Also provided is a composition comprising a glycoprotein, such as an antibody, from which the high mannose glycoforms have been reduced or removed or isolated or enriched using an ion exchange based chromatography step.

### BACKGROUND

Polypeptides that are synthesized in a cell frequently undergo post-translational modifications. Most of the polypeptides that are targeted to the endoplasmic reticulum (ER) are glycosylated, that is, sugar residues (or oligosaccharides) are enzymatically added to the polypeptide during a process known as glycosylation. Glycosylated polypeptides are also referred to as glycoproteins. In eukaryotic cells, a specific oligosaccharide (comprising 14 N-acetyl-glucosamine, mannose, and glucose residues) is attached to arginine residues of polypeptides in the lumen of the ER. Because the oligosaccharide is always transferred to the NH₂ group on the side chain of the arginine residue, this oligosaccharide is called an N-linked or asparagine-linked oligosaccharide. N-linked oligosaccharides are added to an asparagine residue that occurs in the sequence Asparagine-X-Serine or Asparagine-X-Threonine, where X is any amino acid except proline. These two sequences (Asn-X-Ser or Asn-X-Thr), thus, function as signals for N-linked glycosylation. In addition, a series of glycosyl transferase enzymes in the Golgi complex can add sugar residues to the hydroxyl side chain (OH) of serine or threonine amino acids in polypeptides, a process known as O-linked glycosylation.

Following attachment of the N-linked oligosaccharide, further modifications of the sugar residues may occur in both the ER and the Golgi complex. In the ER, glucose residues may be trimmed from the oligosaccharide, along with certain mannose residues. In the Golgi complex, a variety of enzymes act to remove mannose residues and/or add other sugar residues, including N-acetylglucosamine, galactose, and sialic acid. Two broad classes of N-linked oligosaccharides are found in mature glycoproteins: the complex oligosaccharides and the high mannose oligosaccharides. High mannose oligosaccharides typically have no new sugars added to them in the Golgi complex. They contain two N-acetylglucosamines and multiple mannose residues, often approaching the number (9) originally present in the oligosaccharide precursor attached to the polypeptide in the ER. Complex oligosaccharides, on the other hand, can contain more than the original two N-acetylglucosamines as well as a variable number of galactose and sialic acid residues and, in certain instances, fucose. Sialic acid residues are unique in that they are the only sugar residue of glycoproteins that bear a net negative charge.

Glycosylation of polypeptides can affect their properties and function. For example, glycosylation of antibody molecules can affect important immune system functions such as complement activation on antigens, clearance from the body, and potency. In IgG molecules, the binding site for C1q, the first component of the complement activation, is localized to C_{H2} domains in the Fc region (Morrison, S. L. et al., (1994) The Immunologist. 2, 119-124). The presence of high mannose oligosaccharides, such as high mannose 5, on an antibody usually leads to faster clearance from the body, decreasing its potency (Wright, A. et al. (1998) Journal of Immunology. 160, 3393-3402). High levels of high mannose 5 glycoforms can be a concern for therapeutic antibodies due to the effect of these glycoforms on clearance, immunogenicity, and efficacy (Pacis et al., (2011) Biotechnology and Bioengineering. 108(10):2348-58). On the other hand, other studies have shown that high mannose antibodies, generated in the presence of a glycosylation inhibitor, have greater ADCC activity and greater affinity for FcγRIIIa (Pacis et al., (2011) Biotechnology and Bioengineering. 108(10):2348-58).

Glycosylation of an antibody molecule typically depends on the cell culture conditions, including the host cell in which the antibody molecule was cultivated and the type of antibody (Raju, T. S. (2003) BioProcess International. 4, 44-53). Current efforts to control the levels of high mannose 5 glycoforms during the manufacture of therapeutic antibodies are directed to manipulating the cell culture process and not the purification process (Pacis et al., (2011) Biotechnology and Bioengineering. 108(10):2348-58). Glycoform levels of antibodies are not typically affected by downstream purification process conditions.

The presence of high mannose glycoform in a therapeutic antibody as an impurity may affect safety or efficacy. However, in cases in which it is intended by design for therapeutic antibodies to trigger the immune system to generate antibodies against the therapeutic, consistently higher amounts of potentially more immunogenic high mannose glycoforms may be preferred. Thus, there exists the need for controlling the levels of high mannose 5 glycoforms during the manufacture of therapeutic antibodies to ensure product quality (safety and efficacy). The present invention describes process strategies that may be used to obtain therapeutic antibodies with controlled levels of high mannose glycoforms, depending on the specific needs of the therapeutic antibody being manufactured.

### SUMMARY

The present disclosure provides methods of using ion exchange chromatography to reduce the amount of high mannose glycoforms in a glycoprotein sample, such as an antibody sample. These methods can be used to produce a glycoprotein composition having very low levels of high mannose glycoforms. Thus, another aspect is directed to a composition comprising a glycoprotein, such as a monoclonal antibody, wherein the glycoprotein comprises at least two N-linked oligosaccharides and wherein less than 2% of the glycoprotein in the composition is a high mannose glycoform.

The present invention discloses a method of reducing the amount of high mannose glycoform of a glycoprotein in a sample which comprises the high mannose glycoform and at least one other glycoform of the glycoprotein. The method comprises loading the sample onto an ion exchange column under conditions that permit retention of the glycoprotein onto the ion exchange column; passing an elution buffer through the ion exchange column to elute the glycoprotein from the ion exchange column, collecting a first one or more fractions that elute from the ion exchange column and comprise at least one other glycoform; and excluding from the first one or more fractions a second one or more fractions that elute from the ion exchange column before or after the first one or more fractions and contain high mannose glycoform, wherein the amount of high mannose glycoform in the first one or more fractions is reduced relative to the amount of high mannose glycoform in the sample before loading onto the ion exchange column.

In some embodiments, the elution buffer may comprise at least one buffer containing salt species such as sodium chloride, sodium sulfate, ammonium sulfate, arginine, among others. The elution buffer may comprise at least one buffer species such as citrate, acetate, sodium phosphate, Tris, or glycine, among others. Elution buffers may be combinations of at least one buffer containing salt species and at least one buffer from the buffer species. Varying the pH conditions between 3.5 and 8.0 can also be potentially used to achieve the high mannose glycoform separation.

In another aspect, the present disclosure provides methods of using ion exchange chromatography to isolate or enrich high mannose glycoforms in a glycoprotein sample, such as an antibody sample. These methods can be used to produce a glycoprotein composition having very concentrated levels of high mannose glycoforms. Thus, another aspect is directed to a composition comprising a glycoprotein, such as a monoclonal antibody, wherein the glycoprotein comprises at least two N-linked oligosaccharides and wherein at least 50% of the glycoprotein in the composition is a high mannose glycoform.

### BRIEF DESCRIPTION OF THE DRAWINGS

The accompanying drawings, which are incorporated in and constitute a part of this specification, illustrate certain embodiments, and together with the written description, serve to explain certain principles of the antibodies and methods disclosed herein.

FIG. 1 shows a simplified physical structure of the mAb 7.159.2 (ATCC Accession Number PTA-7424) antibody with an N-linked glycosylation site on the C_{H}2 domain of the Fc region and the V_{H} domain of the Fab region.

FIG. 2 shows the major oligosaccharides present on the Fc and Fab regions of the mAb 7.159.2 antibody. Man=mannose; GlcNAc=N-acetylglucosamine; Fuc=fucose; Gal=galactose; NANA=N-acetylneuraminic acid, also known as sialic acid; and 2AB=2-aminobenzamide (fluorescent label for glycan analysis).

FIG. 3 shows a chromatogram of the control run of the mAb 7.159.2 antibody on a POROS® HS 50 (Applied Biosystems, Carlsbad, Calif.) with step elution and concentration of the high mannose 5 glycoform (“Man5”) in the products.

FIG. 4 shows the mAb 7.159.2 products from a linear gradient elution run, with the high mannose 5 glycoforms (“M5”) eluting in later fractions of the gradient.

FIG. 5 shows that a linear gradient elution run with the Control Ab1 antibody (IgG1), does not result in separation of the high mannose 5 glycoform from other glycoforms.

FIG. 6 shows that a linear gradient elution run with the Control Ab3 antibody (IgG2), does not result in separation of the high mannose 5 glycoform from other glycoforms.

FIG. 7 shows the retention times and absorbance values for different fractions of a linear gradient elution run with the mAb 7.159.2 antibody. Charge differences were seen in the different fractions, with early fractions being more acidic and later fractions more basic.

FIG. 8 shows the relative proportion of glycoforms present in mAb 7.159.2 linear gradient elution fractions, with the more mature glycoforms (e.g., sialyated glycoforms) eluting in the early fractions and the less mature glycoforms (e.g., high mannose 5 glycoform) eluting in the later fractions.

FIG. 9 shows the amount of sialic acid (NANA) present in mAb 7.159.2 linear gradient elution fractions, with an overlay of a graphical representation of the trend of glycoform removal during cation exchange chromatography.

FIG. 10 shows the separation of high mannose 5 glycoforms following a run of mAb 7.159.2 through a POROS® XS (Applied Biosystems, Carlsbad, Calif.) column.

FIG. 11 shows the separation of high mannose 5 glycoforms following a run of mAb 7.159.2 through a Nuvia™ S (Bio Rad Laboratories, Hercules, Calif.) column.

FIG. 12 shows the separation of high mannose 5 glycoforms following a run of mAb 7.159.2 through an Eshmuno® S (EMD Millipore, Darmstadt, Germany) column.

FIG. 13 shows the separation of high mannose 5 glycoforms following a run of mAb 7.159.2 through a Capto™ S (GE Healthcare Life Sciences, Piscataway, N.J.) column.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

1. Definitions

In order that the present invention may be more readily understood, certain terms are first defined. Additional definitions and embodiments encompassed by the terms and definitions herein are set forth throughout the detailed description.

As used in this application, the term “high mannose glycoform” refers to a glycoprotein, such as an antibody, having an N-linked oligosaccharide, wherein the N-linked oligosaccharide is a high mannose glycan having 5-9 mannose units.

As used in this application, the term “high mannose 5 glycoform” refers to a glycoprotein, such as an antibody, having an N-linked oligosaccharide, wherein the N-linked oligosaccharide is a high mannose glycan having 5 mannose units.

As used in this application, the term “other glycoform” refers to a glycoprotein, such as an antibody, having an N-linked oligosaccharide, wherein the N-linked oligosaccharide is an oligosaccharide other than a high mannose glycan having 5-9 mannose units.

As used in this application, the term “mannose 5 glycan” refers to an N-linked oligosaccharide having 5 mannose units.

It should be noted that, as used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is also understood that wherever embodiments are described herein with the language “comprising,” otherwise analogous embodiments described in terms of “consisting of” and/or “consisting essentially of” are also provided.

2. Glycoproteins

The methods described in this application can be carried out using any glycoprotein having a high mannose glycoform. In one embodiment, the glycoprotein is a therapeutic glycoprotein, preferably one that is administered to humans.

These glycoproteins include, but are not limited to, antibodies, cytokines (including, but not limited to, interferon-α, interferon-β, interferon-γ, and granulocyte-colony stimulating factor) tumor necrosis factor, transforming growth factor β, interleukin 2; coagulation factors (including but not limited to, factor VIII, factor IX, and human protein C); erythropoietin, IGF-binding protein, epidermal growth factor, growth hormone-releasing factor, annexin V fusion protein, angiostatin, vascular endothelial growth factor-2, myeloid progenitor inhibitory factor-1, and osteoprotegerin. In one embodiment, the glycoprotein is a human glycoprotein.

In certain embodiments, it is desirable to detect or quantify the amount of high mannose glycoforms or other complex glycoforms in a sample. Glycoforms can be detected or quantified using any of a variety of conventional assays or detection methods. For example a specific glycoform of interest can be detected using a lectin or antibody that binds specifically to the desired oligosaccharide. A wide variety of oligosaccharide-specific lectins are available commercially (EY Laboratories, San Mateo, Calif.). Alternatively, antibodies to specific N-linked oligosaccharides are available commercially or may be produced using standard techniques. An appropriate lectin or antibody may be conjugated to a reporter molecule, such as a chromophore, fluorophore, radioisotope, or an enzyme having a chromogenic substrate, to facilitate detection using standard screening techniques, such as spectrophotometry, fluorimetry, fluorescence activated cell sorting, or scintillation counting. Alternatively, it may be desirable to analyze isolated N-linked oligosaccharides. In such cases, an enzyme such as endo-β-N-acetylglucosaminidase may be used to cleave the N-linked oligosaccharides from glycoproteins. Isolated N-linked oligosaccharides may then be analyzed by liquid chromatography (e.g. HPLC), mass spectroscopy, or other suitable means.

3. Antibodies

In certain embodiments, the methods described in this application are used to remove or enrich the amount of high mannose glycoforms in an antibody sample. Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain includes an N-terminal variable (V) domain (VL) and a constant (C) domain (CL). Each heavy chain includes an N-terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. Identification and numbering of framework and CDR residues is as described by Chothia et al., Structural determinants in the sequences of immunoglobulin variable domain, J Mol Biol 1998, 278:457-79, which is hereby incorporated by reference in its entirety.

The term “antibody” includes but is not limited to polyclonal, monoclonal, monospecific, polyspecific, non-specific, humanized, human, single-chain, chimeric, synthetic, recombinant, hybrid, mutated, grafted, and in vitro generated antibodies. In a preferred embodiment, the antibody is a monoclonal antibody. In one embodiment, the monoclonal antibody is a human antibody.

The three dimensional structure of antibodies was elucidated through the use of proteolytic enzymes, including papain. Limited digestion with papain, cleaves an antibody into three fragments. Two of the fragments are identical and contain the antigen binding site. These fragments are called Fab fragments, for Fragment antigen binding. The third fragment has no antigen binding activity and is easily crystallized. It is called the Fc fragment, for Fragment crystallizable. The two identical Fab fragments typically correspond to the arms of the antibody, containing the complete light chain and the V_{H} and C_{H}1 domains of the heavy chain. The Fc fragment typically corresponds to the C_{H}2 and C_{H}3 domains of the heavy chain.

In one embodiment, the antibody is a recombinant, monoclonal antibody. The recombinant monoclonal antibody is prepared from a host cell, including, but not limited to, a bacterial cell, a yeast cell, an insect cell, or a mammalian cell. In a preferred embodiment, the host cell is a mammalian cell. In another embodiment, the recombinant monoclonal antibody is a human antibody. In yet another embodiment, the monoclonal antibody is an IgA, IgE, IgD, IgE, or IgG antibody. In a preferred embodiment, the monoclonal antibody is an IgG antibody, including, but not limited to an IgG1 or IgG2 antibody.

In another embodiment, the antibody comprises at least one N-linked glycosylation site on the Fc region of the antibody and at least one N-linked glycosylation site on the Fab region of the antibody. In another embodiment, the antibody has only one N-linked glycosylation site on the Fc region of the antibody and only one N-linked glycosylation site on the Fab region of the antibody (i.e., at total of 3 N-linked glycosylation sites).

In another embodiment, the antibody binds to insulin-like growth factor 2 (IGF II) with cross reactivity to insulin-like growth factor 1 (IGF I), such as those antibodies disclosed in U.S. Published Application 2007/0196376, which is hereby incorporated by reference in its entirety. In certain embodiments, the antibody binds to IGF II with cross reactivity to IGF I and is a monoclonal, human antibody selected from the group consisting of mAb 7.251.3 (ATCC Accession Number PTA-7422), mAb 7.34.1 (ATCC Accession Number PTA-7423), and mAb 7.159.2 (ATCC Accession Number PTA-7424).

In one embodiment, the antibody binds to IGF II with cross reactivity to IGF I and is a monoclonal, human antibody comprising a heavy chain polypeptide having the amino acid sequence of SEQ ID NO: 1 and a light chain polypeptide having the amino acid sequence of SEQ ID NO: 2. In other embodiments, the antibody binds to IGF II with cross reactivity to IGF I and is a monoclonal, human antibody comprising a heavy chain polypeptide and a light chain polypeptide, wherein the heavy chain polypeptide comprises a heavy chain complementarity determining region 1 (CDR1) having the amino acid sequence of “Ser Tyr Tyr Trp Ser” (SEQ ID NO:7), a heavy chain complementarity determining region 2 (CDR2) having the amino acid sequence of “Tyr Phe Phe Tyr Ser Gly Tyr Thr Asn Tyr Asn Pro Ser Leu Lys Ser” (SEQ ID NO:8), and a heavy chain complementarity determining region 3 (CDR3) having the amino acid sequence of “Ile Thr Gly Thr Thr Lys Gly Gly Met Asp Val” (SEQ ID NO:9), and wherein the light chain polypeptide comprises a light chain CDR1 having the amino acid sequence of “Thr Gly Ser Ser Ser Asn Ile Gly Ala Gly Tyr Asp Val His” (SEQ ID NO:10), a light chain CDR2 having the amino acid sequence of “Gly Asn Asn Asn Arg Pro Ser” (SEQ ID NO:11), and a light chain CDR3 having the amino acid sequence of “Gln Ser Phe Asp Ser Ser Leu Ser Gly Ser Val” (SEQ ID NO:12).

In another embodiment, the antibody binds to IGF II with cross reactivity to IGF I and is a monoclonal, human antibody comprising comprises a heavy chain polypeptide having the amino acid sequence of SEQ ID NO:3 and a light chain polypeptide having the amino acid sequence of SEQ ID NO: 4. In other embodiments, the antibody binds to IGF II with cross reactivity to IGF I and is a monoclonal, human antibody comprising a heavy chain polypeptide and a light chain polypeptide, wherein the heavy chain polypeptide comprises a heavy chain CDR1 having the amino acid sequence of “Ser Tyr Tyr Trp Ser” (SEQ ID NO:13), a heavy chain CDR2 having the amino acid sequence of “Tyr Phe Phe Tyr Ser Gly Tyr Thr Asn Tyr Asn Pro Ser Leu Lys Ser” (SEQ ID NO:14), and a heavy chain CDR3 having the amino acid sequence of “Ile Thr Gly Thr Thr Lys Gly Gly Met Asp Val” (SEQ ID NO:15), and wherein the light chain polypeptide comprises a light chain CDR1 having the amino acid sequence of “Thr Gly Arg Ser Ser Asn Ile Gly Ala Gly Tyr Asp Val His” (SEQ ID NO:16), a light chain CDR2 having the amino acid sequence of “Gly Asn Ser Asn Arg Pro Ser” (SEQ ID NO:17), and a light chain CDR3 having the amino acid sequence of “Gln Ser Tyr Asp Ser Ser Leu Ser Gly Ser Val” (SEQ ID NO:18).

In yet another embodiment, the antibody binds to IGF II with cross reactivity to IGF I and is a monoclonal, human antibody comprising comprises a heavy chain polypeptide having the amino acid sequence of SEQ ID NO:5 and a light chain polypeptide having the amino acid sequence of SEQ ID NO:6. In other embodiments, the antibody binds to IGF II with cross reactivity to IGF I and is a monoclonal, human antibody comprising a heavy chain polypeptide and a light chain polypeptide, wherein the heavy chain polypeptide comprises a heavy chain CDR1 having the amino acid sequence of “Ser Tyr Asp Ile Asn” (SEQ ID NO:19), a heavy chain CDR2 having the amino acid sequence of “Trp Met Asn Pro Asn Ser Gly Asn Thr Gly Tyr Ala Gln Lys Phe Gln Gly” (SEQ ID NO:20), and a heavy chain CDR3 having the amino acid sequence of “Asp Pro Tyr Tyr Tyr Tyr Tyr Gly Met Asp Val” (SEQ ID NO:21), and wherein the light chain polypeptide comprises a light chain CDR1 having the amino acid sequence of “Ser Gly Ser Ser Ser Asn Ile Glu Asn Asn His Val Ser” (SEQ ID NO:22), a light chain CDR2 having the amino acid sequence of “Asp Asn Asn Lys Arg Pro Ser” (SEQ ID NO:23), and a light chain CDR3 having the amino acid sequence of “Glu Thr Trp Asp Thr Ser Leu Ser Ala Gly Arg Val” (SEQ ID NO:24).

Lower amounts of high mannose glycoform in a therapeutic antibody may result in lower immunogenicity, resulting in better efficacy. This may be caused by the generation of less auto antibodies against the therapeutic antibody. And preferred for therapeutic antibodies targeted to neutralize specific antigens or replace proteins, resulting in better dosing. However, for therapeutic antibodies that trigger the immune system to generate antibodies, higher amounts of high mannose glycoforms may be preferred.

A therapeutic antibody having lower high mannose levels will have a longer half-life and may be more efficacious. High mannose glycoform levels in therapeutic antibodies that trigger the immune system to generate antibodies may be more potent.

4. Ion-Exchange Chromatography

The methods of the invention are applicable to ion exchange chromatography.

Ion-exchange chromatography refers to a chromatographic process in which an ionizable solute of interest (for example, a protein of interest in a mixture) interacts with an oppositely charged ligand linked (for example, by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the medium, faster or slower than the solute of interest. Ion-exchange chromatography specifically includes cation exchange, anion exchange, and mixed mode chromatographies.

Cation exchange media refer to a solid phase which is negatively charged, and which has free cations for exchange with cations in an aqueous solution passed over or through the solid phase. Any negatively charged ligand attached to the solid phase suitable to form the cation exchange medium can be used, for example, a carboxylate, sulfonate and others as described below. Commercially available cation exchange media include, but are not limited to, for example, those having a sulfonate based group (for example, MonoS, MiniS, Source 15S and 30S, SP Sepharose Fast Flow, SP Sepharose High Performance from GE Healthcare, Toyopearl SP-650S and SP-650M from Tosoh, Macro-Prep High S from BioRad, Ceramic HyperD 5, Trisacryl M and LS SP and Spherodex LS SP from Pall Technologies); a sulfoethyl based group (for example, Fractogel SE, from EMD, POROS® S- and POROS® S-20 from Applied Biosystems); a sulphopropul based group (for example, TSK Gel SP 5PW and SP-5PW-HR from Tosoh, POROS® HS-20 and HS-50 from Applied Biosystems); a sulfoisobutyl based group (for example, (Fractogel EMD SO.sub.3 from EMD); a sulfoxyethyl based group (for example, SE52, SE53 and Express-Ion S from Whatman), a carboxymethyl based group (for example, CM Sepharose Fast Flow from GE Healthcare, Hydrocell CM from Biochrom Labs Inc., Macro-Prep CM from BioRad, Ceramic HyperD CM, Trisacryl M CM, Trisacryl LS CM, from Pall Technologies, Matrix Cellufine C500 and C200 from Millipore, CM52, CM32, CM23 and Express Ion C from Whatman, Toyopearl CM-650S, CM-650M and CM-650C from Tosoh); sulfonic and carboxylic acid based groups (for example BAKERBOND Carboxy-Sulfon from J. T. Baker); a carboxylic acid based group (for example, WP CBX from J. T Baker, DOWEX MAC-3 from Dow Liquid Separations, Amberlite Weak Cation Exchangers, DOWEX Weak Cation Exchanger, and Diaion Weak Cation Exchangers from Sigma-Aldrich and Fractogel EMD COO—from EMD); a sulfonic acid based group (e.g., Hydrocell SP from Biochrom Labs Inc., DOWEX Fine Mesh Strong Acid Cation Resin from Dow Liquid Separations, UNOsphere 5, WP Sulfonic from J. T. Baker, Sartobind S membrane from Sartorius, Amberlite Strong Cation Exchangers, DOWEX Strong Cation and Diaion Strong Cation Exchanger from Sigma-Aldrich); and a orthophosphate based group (for example, P11 from Whatman).

Anion exchange media refer to a solid phase which is positively charged, and which has free anions for exchange with anions in an aqueous solution passed over or through the solid phase. The functional groups of anion exchange media are typically tertiary or quaternary amino groups and include diethylaminoethyl (DEAE) groups, quaternary aminoethyl groups and quaternary ammonium groups. Matrices include agarose beads, dextran beads, polystyrene beads, and other matrices. Examples of commercially available (e.g., from Amersham Biosciences, now GE Healthcare, and Sigma-Aldrich) anion exchange media include DEAE-SEPHAROSE, Q SEPHAROSE and others. Other suitable anion-exchange chromatography materials, as well as the selection and use of these materials for the present application, are conventional in the art.

Mixed-mode media refer to solid phase materials which typically contain a combination of multiple binding modes like ion exchange, hydrogen bonding, and hydrophobic interactions. Examples of commercially available (e.g., from Pall Corporation, GE Healthcare, and Bio-Rad) mixed-mode media include PPA Hypercel, HEA Hypercel, MEP Hypercel, Capto MMC, CaptoAdhere, and Nuvia cPrime. If the mixed-mode media are operated only utilizing their ion-exchange properties, they are likely to achieve separation of man5 glycoforms.

In some embodiments, the elution buffer used in the chromatography methods of the invention may comprise at least one buffer containing salt species such as sodium chloride, sodium sulfate, ammonium sulfate, arginine, among others. The elution buffer may comprise at least one buffer species such as citrate, acetate, sodium phosphate, Tris, or glycine, among others. Elution buffers may be combinations of at least one buffer containing salt species and at least one buffer from the buffer species. Varying the pH conditions between 3.5 and 8.0 can also be potentially used to achieve the high mannose glycoform separation.

5. Methods of Removing High Mannose Glycoforms from a Sample

The methods described in this application can be used to reduce or remove high mannose glycoforms from a sample containing a glycoprotein. In one embodiment, the glycoprotein is an antibody.

The methods can be used, for example, as a polishing step in the production of a monoclonal antibody. Antibodies are typically produced using cultured mammalian host cells to promote proper folding and glycosylation of the antibody. In recent years, significant improvements have been made in cell culture technology, including advances in cell culture media and feeding strategies, resulting in high cell culture titers of greater than 2 g/L. The cell culture titers may be greater than 2 g/L, greater than 3 g/L, greater than 4 g/L, greater than 5 g/L, or any fraction thereof. The efficient recovery and purification of antibodies from cell culture media is an important part of the antibody production process. A common technique for purifying antibodies from bulk culture media involves an initial purification step using Protein A affinity chromatography, a highly selective process that can result in greater than 95% purity starting from complex cell culture media. Following the capture step (e.g., Protein A), trace levels of process-related contaminants, such as host cell proteins, DNA, leached Protein A, endotoxins, and some cell culture media additives, as well as product-related impurities, such as higher molecular weight aggregates and lower molecular weight degradation products remain with the antibody. These contaminants and impurities can be removed through additional chromatography techniques and are commonly referred to as polishing steps because they reduce trace level contaminants and impurities to levels that are considered safe for therapeutic administration. Prior to Applicant's discovery, however, the polishing step was never considered or used as a way to separate the high mannose glycoforms from other glycoforms present in a monoclonal antibody sample.

Thus, one embodiment is directed to a method of reducing the amount of a high mannose glycoform of a glycoprotein in a sample, wherein the sample comprises the high mannose glycoform and at least one other glycoform of the glycoprotein, the method comprising: (a) loading the sample onto an ion exchange column under conditions that permit the retention of the glycoprotein onto the ion exchange column; (b) passing an elution buffer through the ion exchange column to elute the glycoprotein from the ion exchange column; (c) collecting a first one or more fractions that elute from the ion exchange column and comprise the at least one other glycoform; and (d) excluding from the first one or more fractions a second one or more fractions, wherein the second one or more fractions elute from the ion exchange column before or after the first one or more fractions and contain the high mannose glycoform, and wherein the amount of the high mannose glycoform in the first one or more fractions is reduced relative to the amount of the high mannose glycoform in the sample before loading onto the ion exchange column.

In one embodiment, the ion exchange column is a cation exchange column and the second one or more fractions that contain the high mannose glycoform elute from the cation exchange column after the first one or more fractions that contain the at least one other glycoform.

In another embodiment, the ion exchange column is an anion exchange column and the second one or more fractions that contain the high mannose glycoform elute from the anion exchange column before the first one or more fractions that contain the at least one other glycoform.

In yet another embodiment, the method further comprises a step of measuring the amount of the high mannose glycoform in the first one or more fractions or the second one or more fractions. In another embodiment, the method further comprises a step of washing the ion exchange column after loading the sample and before passing the elution buffer through the ion exchange column.

In one embodiment, the glycoprotein is an antibody, including, but not limited to those described above in the “Antibodies” section, or elsewhere, in this application.

In another embodiment, the amount of the high mannose glycoform of the monoclonal antibody in the first one or more fractions is less than 2% of the total amount of monoclonal antibody. In another embodiment, the amount of the high mannose glycoform of the monoclonal antibody in the first one or more fractions is less than 1% of the total amount of monoclonal antibody. In yet another embodiment, the amount of the high mannose glycoform of the monoclonal antibody in the first one or more fractions is reduced at least 100-, 50-, 10-, 9-, 8-, 7-, 6-, 5-, 4-, 3-, or 2-fold relative to the amount of high mannose glycoform of the monoclonal antibody in the sample before loading onto the ion exchange column.

6. Methods of Isolating High Mannose Glycoforms from a Sample

The methods described in this application can also be used to isolate or enrich high mannose glycoforms in a sample containing a glycoprotein. In one embodiment, the glycoprotein is an antibody.

Thus, one embodiment is directed to a method of isolating a high mannose glycoform of a glycoprotein in a sample, wherein the sample comprises the high mannose glycoform and at least one other glycoform of the glycoprotein, the method comprising: (a) loading the sample onto an ion exchange column under conditions that permit the retention of the glycoprotein onto the ion exchange column; (b) passing an elution buffer through the ion exchange column to elute the glycoprotein from the ion exchange column; (c) collecting a first one or more fractions that elute from the ion exchange column and contain the high mannose glycoform; and (d) excluding from the first one or more fractions a second one or more fractions, wherein the second one or more fractions elute from the ion exchange column before or after the first one or more fractions and contain the at least one other glycoform, thereby isolating the high mannose glycoform in the sample.

In one embodiment, the ion exchange column is a cation exchange column and the first one or more fractions that contain the high mannose glycoform elute from the cation exchange column after the second one or more fractions that contain the at least one other glycoform.

In another embodiment, the ion exchange column is an anion exchange column and the first one or more fractions that contain the high mannose glycoform elute from the anion exchange column before the second one or more fractions that contain the at least one other glycoform.

In yet another embodiment, the method further comprises a step of measuring the amount of the high mannose glycoform in the first one or more fractions or the second one or more fractions. In another embodiment, the method further comprises a step of washing the ion exchange column after loading the sample and before passing the elution buffer through the ion exchange column.

In one embodiment, the glycoprotein is an antibody, including, but not limited to, those described above in the “Antibodies” section, or elsewhere, in this application.

In another embodiment, the amount of the high mannose glycoform of the monoclonal antibody in the first one or more fractions is increased 10-, 20-, 30, 40-, 50-, 60-, 70-, 80-, 90-, 100-, 200-, or greater than 200-fold relative to the amount of high mannose glycoform of the monoclonal antibody in the sample before loading onto the ion exchange column.

7. Compositions

The methods described in this application provide a mechanism for reducing or removing high mannose glycoforms from a glycoprotein sample, resulting in compositions having very low levels of high mannose glycoforms.

Thus, another aspect is directed to a composition comprising a glycoprotein, such as a monoclonal antibody, wherein the glycoprotein comprises at least two N-linked oligosaccharides and wherein less than 2% of the glycoprotein in the composition is a high mannose glycoform. In one embodiment, less than 1% of the glycoprotein in the composition is a high mannose glycoform. In one embodiment, the glycoprotein is an antibody, including, but not limited to, those described above in the “Antibodies” section, or elsewhere, in this application.

The methods described in this application also provide a mechanism for isolating or enriching high mannose glycoforms from a glycoprotein sample, resulting in compositions having very concentrated levels of high mannose glycoforms.

Thus, another aspect is directed to a composition comprising a glycoprotein, such as a monoclonal antibody, wherein the glycoprotein comprises at least two N-linked oligosaccharides and wherein at least 10%, 20%, 30%, 40%, 50%, 60%, 70%, 80%, 90%, or 95% of the glycoprotein in the composition is a high mannose glycoform. In one embodiment, the glycoprotein is an antibody, including, but not limited to, those described above in the “Antibodies” section, or elsewhere, in this application.

### EXAMPLES

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### Example 1

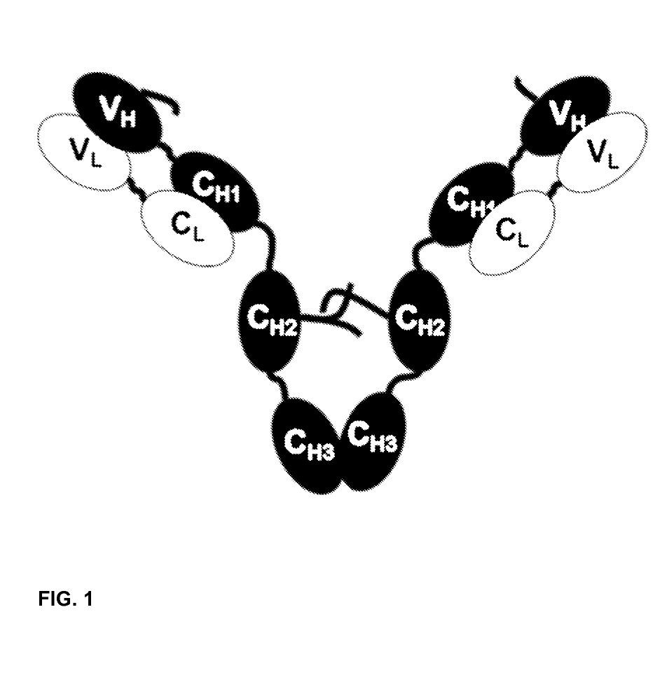

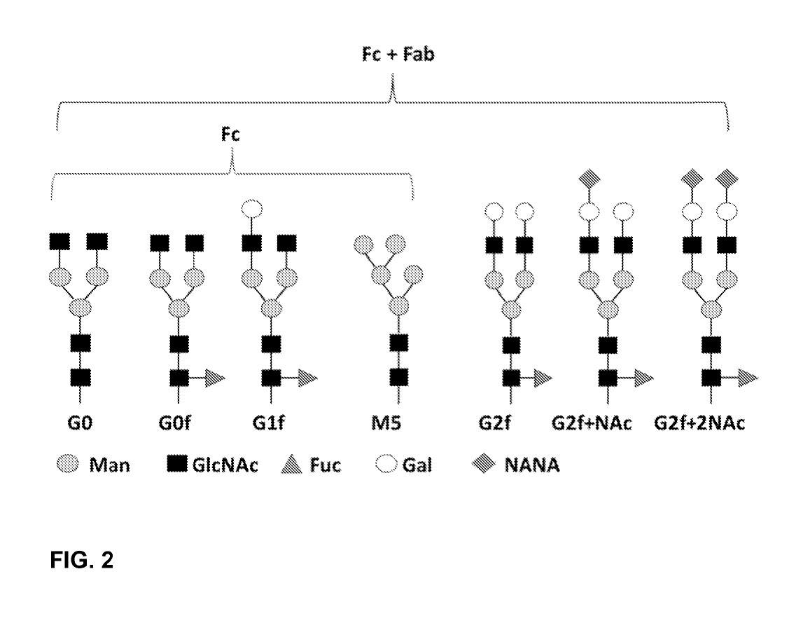

The mAb 7.159.2 (ATCC Accession Number PTA-7424) is a fully human IgG22, monoclonal antibody molecule composed of two identical heavy chains and two identical light chains, with an overall molecular weight of approximately 151 kDa. The extinction coefficient is 1.54 (mg/mL)⁻¹cm⁻¹ and the pI is between 7.8 and 8.8. There are two N-linked oligosaccharide glycosylation sites, one in the heavy chain in the Fab region at residue Asn-73 and one in the Fc region at residue Asn-296 (FIG. 1). The oligosaccharides at both sites are predominantly of the complex type. Depending on the cell clone and bioreactor conditions, the high mannose 5 glycoforms of mAb 7.159.2 can vary between about 3-11% (data not shown). The glycans present on CH₂ domains in the Fc region and on V_{H} domains in the Fab region are shown in FIG. 2. The Fab region has higher proportions of mature sialylated glycans that are acidic in nature, in addition to other glycans present on both Fc and Fab regions. The Fc region has a higher proportion of less mature glycoforms, such as the high mannose 5 glycoform.

The POROS® HS 50 (Applied Biosystems, Carlsbad, Calif.) cation-exchange chromatography was used as a polishing step to remove process- and product-related impurities in the mAb 7.159.2 purification process. Analysis of the chromatography load and products showed that the column effectively removed both aggregates and high mannose 5 glycoforms (“M5”) of the antibody (Table 1). Therefore, experiments were conducted to further understand the mechanism behind the removal of the high mannose 5 glycoforms.

TABLE 1
Removal of aggregate and M5 glycoforms
across ion exchange chromatography step

| | | | |
|---|---|---|---|
| Cell Culture Scale | Name | Aggregate (%) | M5 (%) |

| 2000 | L | HS 50 Load | 3.3 | 5.8 |
|---|---|---|---|---|
| | | Product | 0.2 | 2.1 |
| 20 | L | HS 50 Load | 2.4 | 4.2 |
| | | Product | 0.3 | 1.1 |
| 100 | L | HS 50 Load | 2.7 | 4.4 |

| | | |
|---|---|---|
| Product | 0.3 | 0.7 |

Linear gradient elution (LGE) studies were performed with a 1.1 cm×17.3 cm (diameter×height) POROS® HS 50 (Applied Biosystems, Carlsbad, Calif.) column under conditions listed below in Table 1B:

TABLE 1B
Column Conditions

| | |
|---|---|
| Column Volume | 16.4 mL |
| Linear Velocity | 300 cm/hr |
| Loading Capacity | 15 mg/mL |
| Pre-Equilibration | 100 mM sodium phosphate, 1M sodium |
| | chloride, pH 7.0, 4CV |
| Equilibration | 10 mM sodium citrate, pH 5.5, 3CV |
| Wash | 10 mM sodium citrate, pH 5.5, 5CV |
| Elution | 20 mM sodium citrate, 0-200 mM |
| | sodium chloride, pH 5.4, 20CV linear |
| | gradient |
| Strip | 100 mM sodium phosphate, 1M sodium |
| | chloride, pH 7.0, 3CV |

During the LGE studies with mAb 7.159.2 and other IgG antibodies (Control Ab1, Control Ab2 and Control Ab3), 0.5CV fractions were collected and analyzed using A280 nm, high pressure size exclusion chromatography (HPSEC) and oligosaccharide profile analyses to determine the change in monomer, aggregate, and high mannose 5 glycoform concentrations across the fractions. As a control run, a step elution run was performed with mAb 7.159.2 as load and the product was eluted using 20 mM sodium citrate, 39 mM sodium chloride, pH 5.4.

Preparative scale size-exclusion chromatography (SEC) was performed using mAb 7.159.2, to isolate and characterize the aggregates and monomers by oligosaccharide analysis. Fractions from mAb 7.159.2 LGE runs were also analyzed with analytical ion exchange chromatography (IEC) and sialic acid assay.

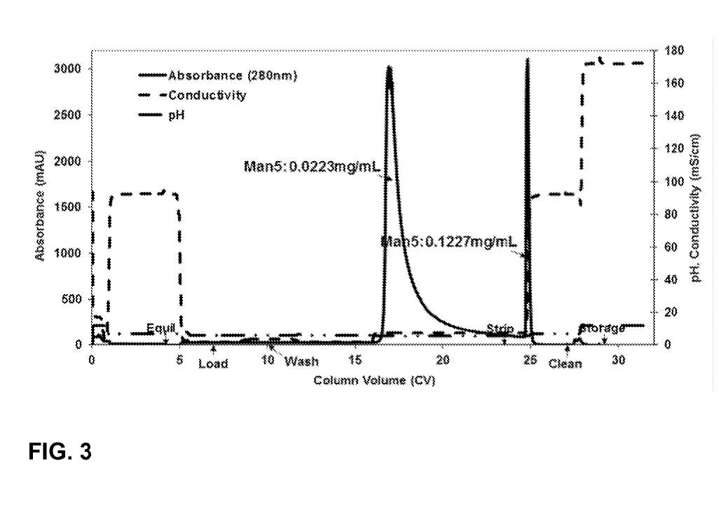

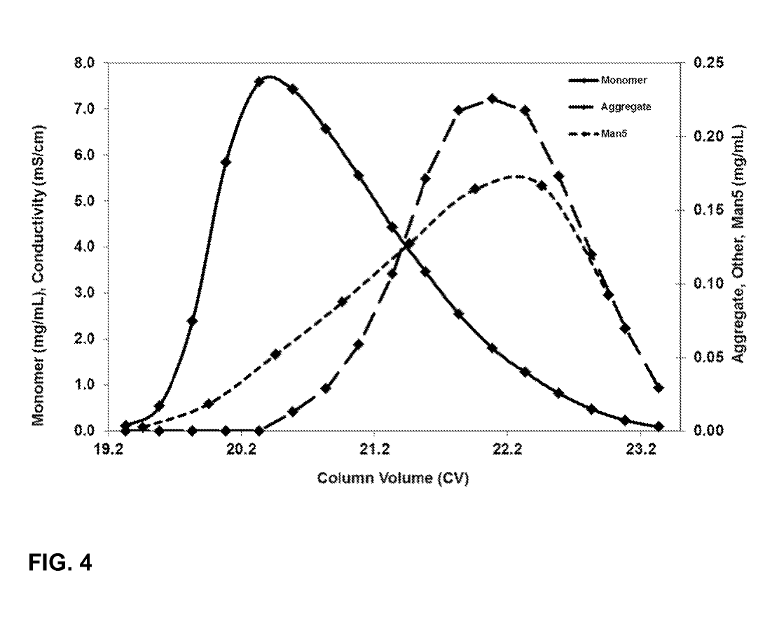

Analysis of POROS® HS 50 (Applied Biosystems, Carlsbad, Calif.) fractions from the control run with step elution showed that the concentration of high mannose 5 glycoforms in the strip was about five-fold greater than that in the elution (FIG. 3), indicating that high mannose 5 glycoforms get removed during the strip. HPSEC and oligosaccharide profile analyses of mAb 7.159.2 fractions from the LGE run confirmed that high mannose 5 glycoforms elute in later fractions of gradient (FIG. 4).

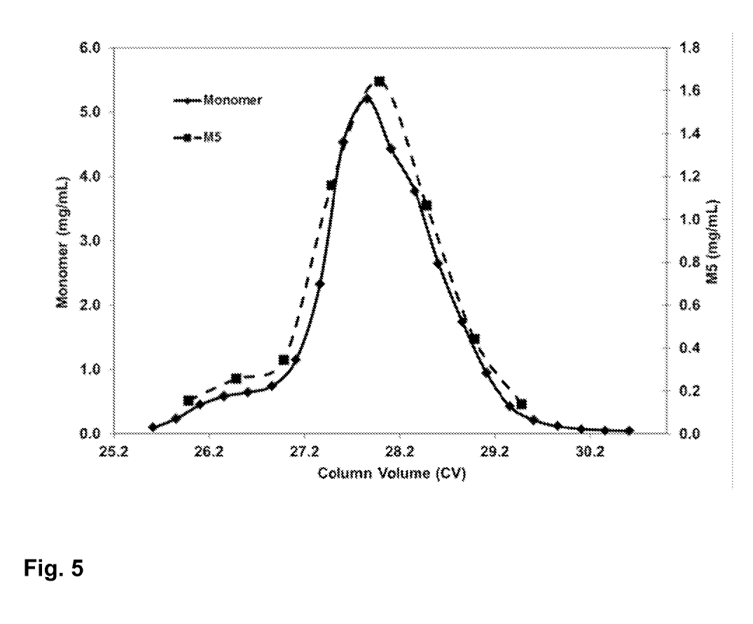

LGE runs were performed with Control Ab1 (IgG1) and Control Ab2 (IgG1), which contain 33% and 2% high mannose glycoforms, respectively. As shown in FIG. 5, oligosaccharide profile analysis for Control Ab1 indicates that there was no separation of high mannose 5 glycoforms. Control Ab2 also showed a similar profile (data not shown), thus, suggesting that the initial high mannose 5 glycoform content does not affect its removal during the ion exchange chromatography step.

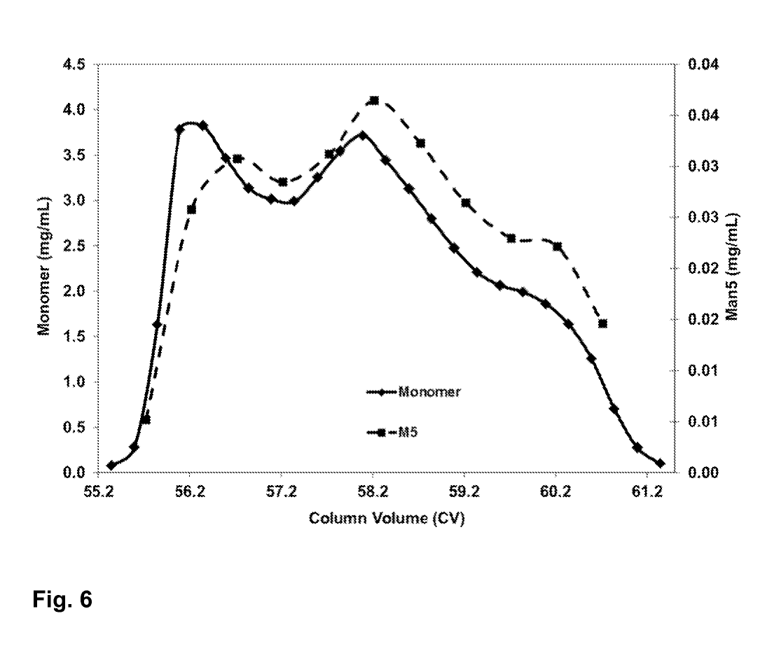

To test whether the removal of the high mannose 5 glycoform was only observed with IgG2 antibodies, an LGE run was performed with Control Ab3 (IgG2). As shown in FIG. 6, the run with Control Ab3 did not result in removal of the high mannose 5 glycoforms, indicating that this behavior was not a specific property of IgG2 antibodies.

Analysis of mAb 7.159.2 LGE fractions showed that there was a corresponding increase in aggregates with increase in high mannose 5 glycoforms, suggesting that high mannose 5 glycoform removal might be related to aggregate removal. SEC was performed to isolate the aggregates and monomers. Oligosaccharide analysis showed that the high mannose 5 glycoform levels in monomers and aggregates are 2.9% and 2.3% respectively, indicating that aggregates and high mannose 5 glycoform removal are not related.

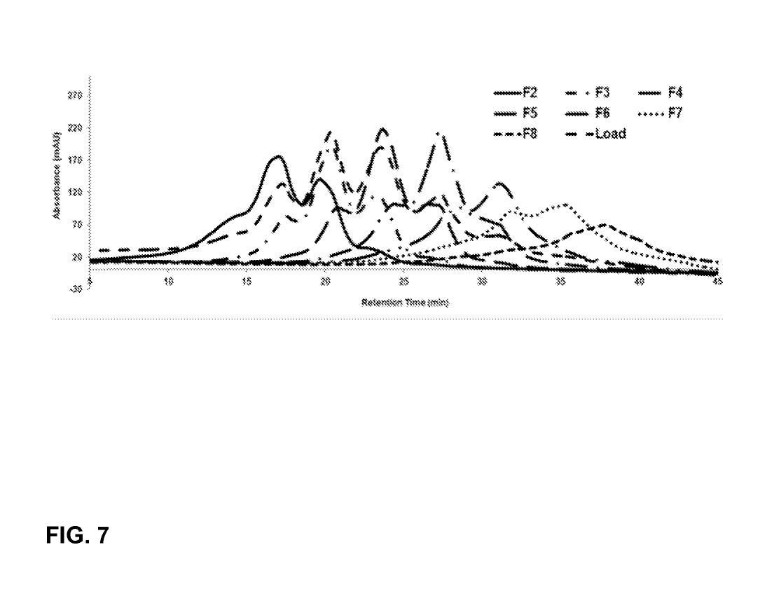

Without intending to be bound by any theory, it appears that removal of mAb 7.159.2 high mannose 5 glycoforms during ion exchange chromatography is likely due to the charge differences on the antibody molecules (FIG. 7). More mature glycoforms that contain sialic acid in the Fab region are relatively more acidic and are removed in the early fractions of elution. The less Fab-sialylated and hence less mature and more basic glycoforms get removed during the later fractions of elution. This behavior was not found in Control Ab1, Control Ab2, and Control Ab3 antibody molecules as they do not have a glycosylation site on the Fab region and are not Fab-sialylated. It is likely in the case of mAb 7.159.2, the less Fab-sialylated (less mature) glycoforms that elute in the later fractions also contain higher proportions of the high mannose 5 glycoforms in the Fc region.

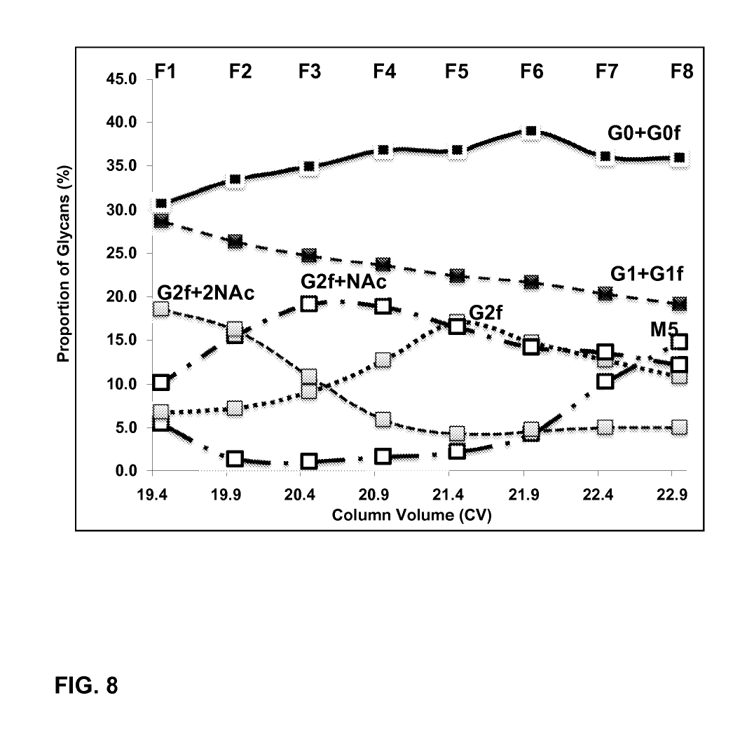

FIG. 8 provides an analysis of the oligosaccharides data to help understand the separation of glycoforms across LGE fractions. The results show that the proportion of more processed glycoforms, such as the sialyated glycoforms, is higher in the earlier fractions of elution and the proportion of less processed glycoforms increases in the later fractions. The G2f+2NAc, G2f+NAc and G2f glycoforms have distinct peaks across the elution profile with the more acidic glycoforms eluting earlier and less acidic glycoforms eluting later. The proportion of high mannose 5 glycoforms increases with the decrease in proportions of G2f+2NAc, G2f+NAc and G2f. The proportion of other less mature glycoforms GO+GOf (FIG. 8) also increases with the decrease in proportions of G2f+2NAc, G2f+NAc and G2f.

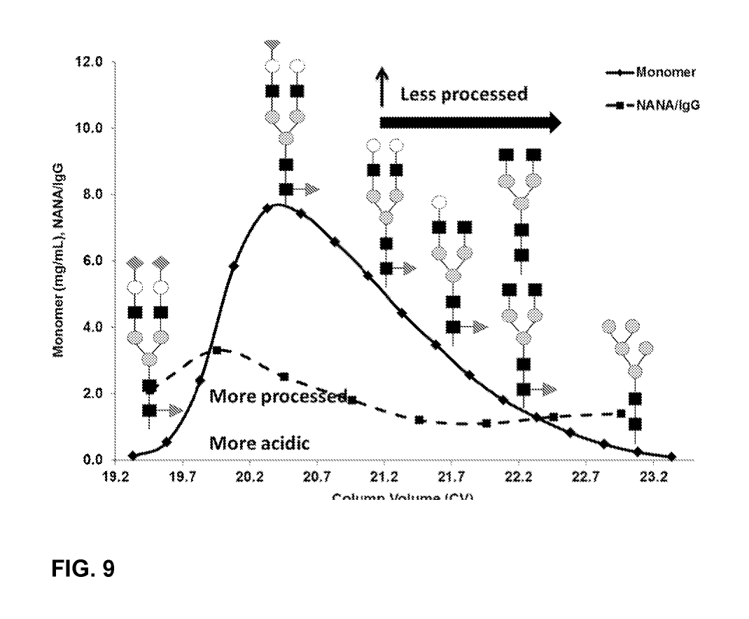

A sialic acid assay was performed to monitor the sialic acid changes in mAb 7.159.2 fractions. Results from the sialic acid assay show that the number of sialic acid per molecule is higher in the early fractions, consistent with the oligosaccharide data showing that the more Fab-sialylated glycoforms elute in the earlier fractions and less mature glycoforms elute in the later fractions (FIG. 9).

The study results indicate that high mannose glycoforms are removed during the mAb 7.159.2 cation-exchange chromatography step due to charge separation created by the Fab-sialylated glycoforms. The results also suggest that ion-exchange chromatography can be used to separate sialylated glycoforms, high-mannose glycoforms, and other less mature glycoforms.

### Example 2

### High Mannose 5 Removal Across Multiple Scales

Oligosaccharide analysis of the POROS® HS 50 (Applied Biosystems, Carlsbad, Calif.) load material (mAb 7.159.2) and elution products showed that high mannose 5 glycoforms were reduced from 4.2-6.0% to 0.7-2.1% across multiple scales (20 L lot, 100 L lot, 500 L lot, and 2000 L lot). Peptide mapping analysis also showed that high mannose 5 glycoforms were reduced from 4.7-4.9% to 0.6-1.1% by the POROS® HS 50 (Applied Biosystems, Carlsbad, Calif.) column step.

### Example 3

### High Mannose 5 Removal with Other Ion Exchange Columns

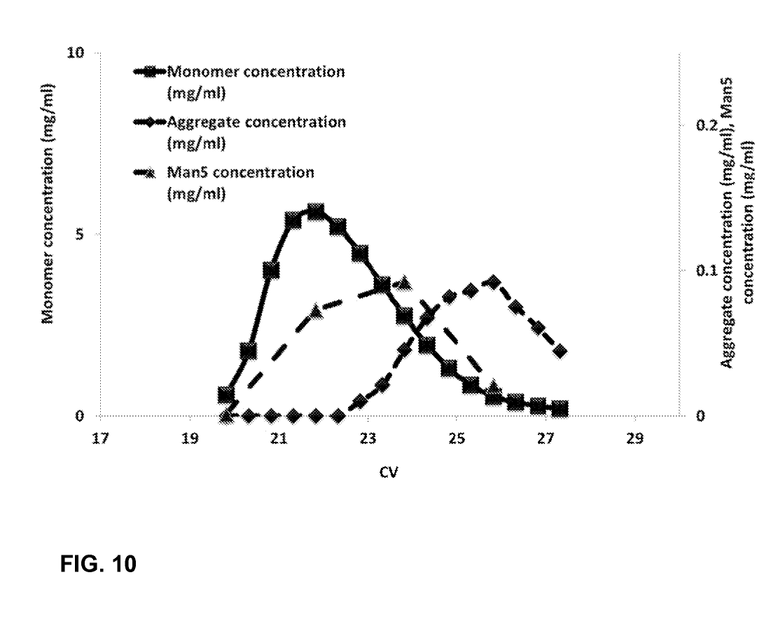

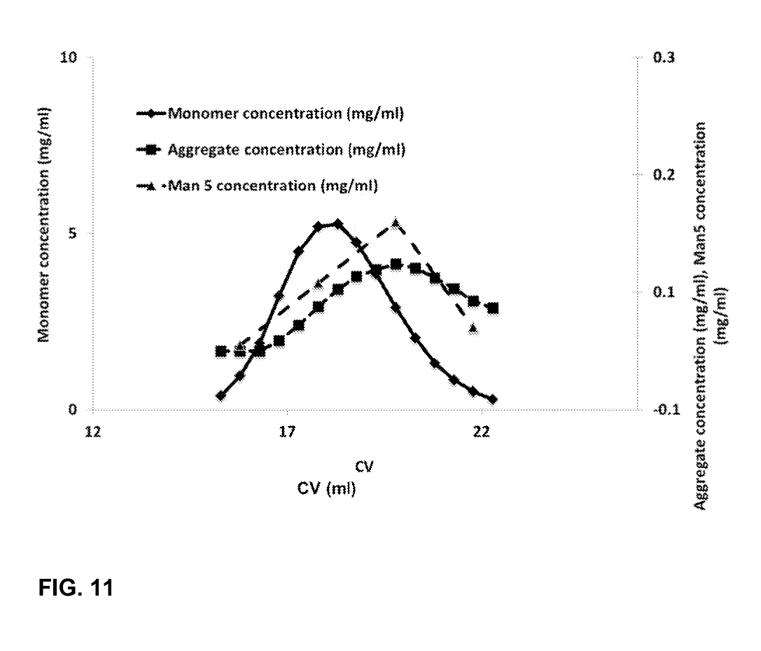

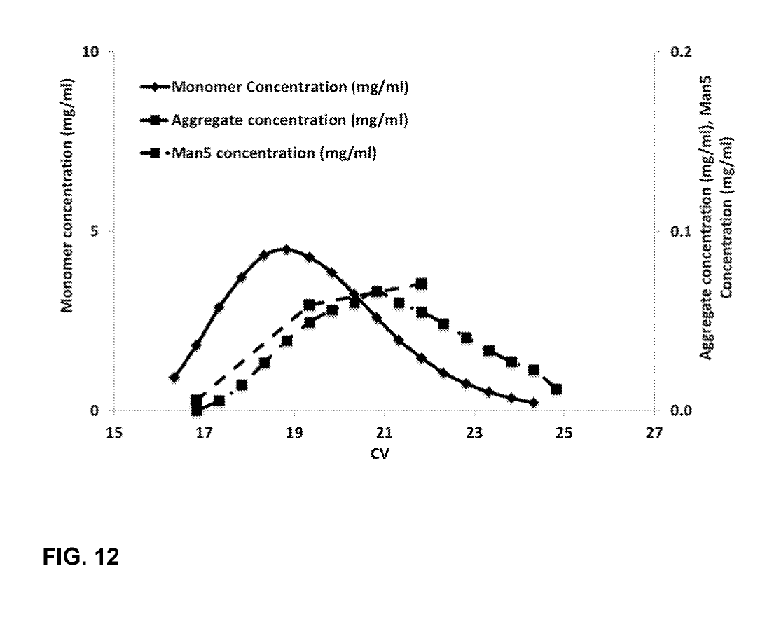

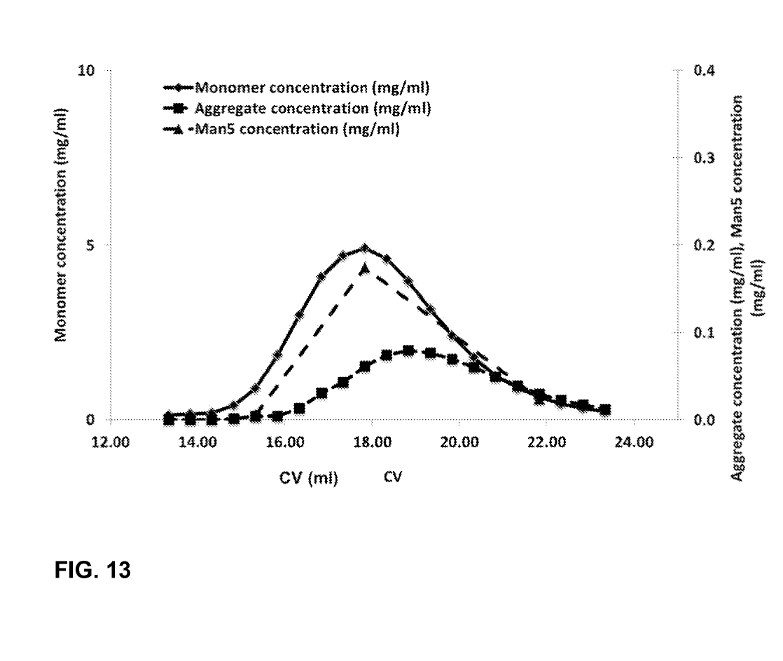

Other cation exchange chromatography resins, including POROS® XS (Applied Biosystems, Carlsbad, Calif.), Nuvia™ S (Bio Rad Laboratories, Hercules, Calif.), Eshmuno® S (EMD Millipore, Darmstadt, Germany), and Capto™ S (GE Healthcare Life Sciences, Piscataway, N.J.) were tested and also showed separation of high mannose 5 glycoforms to different degrees (FIGS. 10-13). Of the additioal columns tested, Capto™ S (GE Healthcare Life Sciences, Piscataway, N.J.) showed the least amount of separation of the high mannose 5 glycoform.

### Sequence Listing

```
SEQ ID NO: 1
Gln Val Gln Leu Gln Glu Ser Gly Pro Gly Leu Val Lys Pro Ser Glu
  1 5 10 15
Thr Leu Ser Leu Thr Cys Thr Val Ser Gly Gly Ser Ile Ser Ser Tyr
                        20 25 30
Tyr Trp Ser Trp Ile Arg Gln Pro Pro Gly Lys Gly Leu Glu Trp Ile
                35 40 45
Gly Tyr Phe Phe Tyr Ser Gly Tyr Thr Asn Tyr Asn Pro Ser Leu Lys
        50 55 60
Ser Arg Val Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                                85 90 95
Cys Ile Thr Gly Thr Thr Lys Gly Gly Met Asp Val Trp Gly Gln Gly
                                100 105 110
Thr Thr Val Thr Val Ser Ser Ala
                115 120
SEQ ID NO: 2
Gln Ser Val Leu Thr Gln Pro Pro Ser Val Ser Gly Ala Pro Gly Gln
  1 5 10 15
Arg Val Thr Ile Ser Cys Thr Gly Ser Ser Ser Asn Ile Gly Ala Gly
                        20 25 30
Tyr Asp Val His Trp Tyr Gln Gln Leu Pro Gly Thr Ala Pro Lys Leu
                35 40 45
Leu Ile Tyr Gly Asn Asn Asn Arg Pro Ser Gly Val Pro Asp Arg Phe
        50 55 60
Ser Gly Ser Lys Ser Gly Thr Ser Ala Ser Leu Ala Ile Thr Gly Leu
65 70 75 80
Gln Ala Asp Asp Glu Ala Asp Tyr Tyr Cys Gln Ser Phe Asp Ser Ser
                                85 90 95
Leu Ser Gly Ser Val Phe Gly Gly Gly Thr Lys Leu Thr Val Leu Gly
                        100 105 110
SEQ ID NO: 3
Gln Val Gln Leu Gln Glu Ser Gly Pro Gly Leu Val Lys Pro Ser Glu
  1 5 10 15
Thr Leu Ser Leu Thr Cys Thr Val Ser Gly Gly Ser Ile Ser Ser Tyr
                        20 25 30
Tyr Trp Ser Trp Ile Arg Gln Pro Pro Gly Arg Gly Leu Glu Trp Ile
                35 40 45
Gly Tyr Phe Phe Tyr Ser Gly Tyr Thr Asn Tyr Asn Pro Ser Leu Lys
        50 55 60
Ser Arg Val Thr Met Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                                85 90 95
Cys Ile Thr Gly Thr Thr Lys Gly Gly Met Asp Val Trp Gly Gln Gly
                        100 105 110
Ala Thr Val Thr Val Ser Ser Ala
                115 120
SEQ ID NO: 4
Gln Ser Val Leu Thr Gln Ala Pro Ser Val Ser Gly Ala Pro Gly Gln
  1 5 10 15
Arg Val Thr Ile Ser Cys Thr Gly Arg Ser Ser Asn Ile Gly Ala Gly
                        20 25 30
Tyr Asp Val His Trp Tyr Gln Gln Phe Pro Gly Thr Ala Pro Lys Leu
                35 40 45
Leu Ile Tyr Gly Asn Ser Asn Arg Pro Ser Gly Val Pro Asp Arg Phe
        50 55 60
Ser Gly Ser Lys Ser Gly Thr Ser Ala Ser Leu Ala Ile Thr Gly Leu
65 70 75 80
Gln Ala Glu Asp Glu Ala Asp Tyr Tyr Cys Gln Ser Tyr Asp Ser Ser
                                85 90 95
Leu Ser Gly Ser Val Phe Gly Gly Gly Thr Lys Leu Thr Val Leu Gly
                        100 105 110
SEQ ID NO: 5
Gln Val Gln Leu Val Gln Ser Gly Ala Glu Val Lys Lys Pro Gly Ala
  1 5 10 15
Ser Val Lys Val Ser Cys Lys Ala Ser Gly Tyr Thr Phe Thr Ser Tyr
                        20 25 30
Asp Ile Asn Trp Val Arg Gln Ala Thr Gly Gln Gly Leu Glu Trp Met
                35 40 45
Gly Trp Met Asn Pro Asn Ser Gly Asn Thr Gly Tyr Ala Gln Lys Phe
        50 55 60
Gln Gly Arg Val Thr Met Thr Arg Asn Thr Ser Ile Ser Thr Ala Tyr
65 70 75 80
Met Glu Leu Ser Ser Leu Arg Ser Glu Asp Thr Ala Val Tyr Tyr Cys
                                85 90 95
Ala Arg Asp Pro Tyr Tyr Tyr Tyr Tyr Gly Met Asp Val Trp Gly Gln
                        100 105 110
Gly Thr Thr Val Thr Val Ser Ser Ala
                115 120
SEQ ID NO: 6
Gln Ser Val Leu Thr Gln Pro Pro Ser Val Ser Ala Ala Pro Gly Gln
  1 5 10 15
Lys Val Thr Ile Ser Cys Ser Gly Ser Ser Ser Asn Ile Glu Asn Asn
                        20 25 30
His Val Ser Trp Tyr Gln Gln Leu Pro Gly Thr Ala Pro Lys Leu Leu
                35 40 45
Ile Tyr Asp Asn Asn Lys Arg Pro Ser Gly Ile Pro Asp Arg Phe Ser
        50 55 60
Gly Ser Lys Ser Gly Thr Ser Ala Thr Leu Gly Ile Thr Gly Leu Gln
65 70 75 80
Thr Gly Asp Glu Ala Asp Tyr Tyr Cys Glu Thr Trp Asp Thr Ser Leu
                                85 90 95
Ser Ala Gly Arg Val Phe Gly Gly Gly Thr Lys Leu Thr Val Leu Gly
                        100 105 110
SEQ ID NO: 7
Ser Tyr Tyr Trp Ser
SEQ ID NO: 8
Tyr Phe Phe Tyr Ser Gly Tyr Thr Asn Tyr Asn Pro Ser Leu Lys Ser
SEQ ID NO: 9
Ile Thr Gly Thr Thr Lys Gly Gly Met Asp Val
SEQ ID NO: 10
Thr Gly Ser Ser Ser AsnIle Gly Ala Gly Tyr Asp Val His
SEQ ID NO: 11
Gly Asn Asn Asn Arg Pro Ser
SEQ ID NO: 12
Gln Ser Phe Asp Ser Ser Leu Ser Gly Ser Val
SEQ ID NO: 13
Ser Tyr Tyr Trp Ser
SEQ ID NO: 14
Tyr Phe Phe Tyr Ser Gly Tyr Thr Asn Tyr Asn Pro Ser Leu Lys Ser
SEQ ID NO: 15
Ile Thr Gly Thr Thr Lys Gly Gly Met Asp Val
SEQ ID NO: 16
Thr Gly Arg Ser Ser Asn Ile Gly Ala Gly Tyr Asp Val His
SEQ ID NO: 17
Gly Asn Ser Asn Arg Pro Ser
SEQ ID NO: 18
Gln Ser Tyr Asp Ser SerLeu Ser Gly Ser Val
SEQ ID NO: 19
Ser Tyr Asp Ile Asn
SEQ ID NO: 20
Trp Met Asn Pro Asn Ser Gly Asn Thr Gly Tyr Ala Gln Lys Phe Gln
Gly
SEQ ID NO: 21
Asp Pro Tyr Tyr Tyr Tyr Tyr Gly Met Asp Val
SEQ ID NO: 22
Ser Gly Ser Ser Ser Asn Ile Glu Asn Asn His Val Ser
SEQ ID NO: 23
Asp Asn Asn Lys Arg Pro Ser
SEQ ID NO: 24
Glu Thr Trp Asp Thr Ser Leu Ser Ala Gly Arg Val
```

What is claimed:

1. A method of reducing the amount of a high mannose glycoform of a glycoprotein in a sample, wherein the sample comprises the high mannose glycoform and at least one other glycoform of the glycoprotein, the method comprising:
(a) loading the sample onto an ion exchange column under conditions that permit the retention of the glycoprotein onto the ion exchange column;
(b) passing an elution buffer through the ion exchange column to elute the glycoprotein from the ion exchange column;
(c) collecting a first one or more fractions that elute from the ion exchange column and comprise the at least one other glycoform; and
(d) excluding from the first one or more fractions a second one or more fractions, wherein the second one or more fractions elute from the ion exchange column before or after the first one or more fractions and contain the high mannose glycoform,
wherein the amount of the high mannose glycoform in the first one or more fractions is reduced relative to the amount of the high mannose glycoform in the sample before loading onto the ion exchange column.

2. The method of claim 1, wherein the ion exchange column is a cation exchange column and wherein the second one or more fractions that contain the high mannose glycoform elute from the cation exchange column after the first one or more fractions that contain the at least one other glycoform.

3. The method of claim 1, wherein the ion exchange column is an anion exchange column and wherein the second one or more fractions that contain the high mannose glycoform elute from the anion exchange column before the first one or more fractions that contain the at least one other glycoform.

4. The method of claim 1, further comprising a step of measuring the amount of the high mannose glycoform in the first one or more fractions or the second one or more fractions.

5. The method of any one of claims 1-4, wherein the glycoprotein is a monoclonal antibody.

6. The method of any one of claim 5, wherein the monoclonal antibody is an IgG antibody.

7. The method of any one of claims 5, wherein the monoclonal antibody is an IgG2 antibody.

8. The method of any one of claims 5-7, wherein the monoclonal antibody is a recombinant monoclonal antibody.

9. The method of claim 8, wherein the recombinant monoclonal antibody is prepared from a host cell, wherein the host cell is a bacterial cell, a yeast cell, an insect cell, or a mammalian cell.

10. The method of any one of claims 5-9, wherein the monoclonal antibody is a human antibody.

11. The method of any one of claims 5-10, wherein the monoclonal antibody comprises at least one N-linked glycosylation site on the Fc region and at least one N-linked glycosylation site on the Fab region.

12. The method of any one of claims 5-11, wherein the high mannose glycoform of the monoclonal antibody is a high mannose 5 glycoform of the monoclonal antibody.

13. The method of any one of claims 5-12, wherein the monoclonal antibody comprises a heavy chain polypeptide having the amino acid sequence of SEQ ID NO:5 and a light chain polypeptide having the amino acid sequence of SEQ ID NO:6.

14. The method of any one of claims 5-12, wherein the monoclonal antibody comprises a heavy chain polypeptide and a light chain polypeptide, wherein the heavy chain polypeptide comprises a heavy chain complementarity determining region (CDR) 1 having the amino acid sequence of (SEQ ID NO:19), a heavy chain CDR2 having the amino acid sequence of (SEQ ID NO:20), and a heavy chain CDR3 having the amino acid sequence of (SEQ ID NO:21), and wherein the light chain polypeptide comprises a light chain CDR1 having the amino acid sequence of (SEQ ID NO:22), a light chain CDR2 having the amino acid sequence of (SEQ ID NO:23), and a light chain CDR3 having the amino acid sequence of (SEQ ID NO:24).

15. The method of any one of claims 5-14, wherein the amount of the high mannose glycoform of the monoclonal antibody in the first one or more fractions is less than 2% of the total amount of monoclonal antibody.

16. The method of any one of claims 5-14, wherein the amount of the high mannose glycoform of the monoclonal antibody in the first one or more fractions is reduced 10-fold relative to the amount of high mannose glycoform of the monoclonal antibody in the sample before loading onto the ion exchange column.

17. The method of any one of claims 1-16, further comprising a step of washing the ion exchange column after loading the sample and before passing the elution buffer through the ion exchange column.

18. A composition comprising a monoclonal antibody, wherein the monoclonal antibody comprises at least two N-linked oligosaccharides and wherein less than 2% of the monoclonal antibody in the composition is a high mannose glycoform.

19. The composition of claim 18, wherein less than 1% of the monoclonal antibody in the composition is a high mannose glycoform.

20. The composition of claim 18 or 19, wherein the monoclonal antibody comprises a heavy chain polypeptide having the amino acid sequence of SEQ ID NO:5 and a light chain polypeptide having the amino acid sequence of SEQ ID NO:6.

21. The composition of claim 18 or 19, wherein the monoclonal antibody comprises a heavy chain polypeptide and a light chain polypeptide, wherein the heavy chain polypeptide comprises a heavy chain complementarity determining region (CDR) 1 having the amino acid sequence of (SEQ ID NO:19), a heavy chain CDR2 having the amino acid sequence of (SEQ ID NO:20), and a heavy chain CDR3 having the amino acid sequence of (SEQ ID NO:21), and wherein the light chain polypeptide comprises a light chain CDR1 having the amino acid sequence of (SEQ ID NO:22), a light chain CDR2 having the amino acid sequence of (SEQ ID NO:23), and a light chain CDR3 having the amino acid sequence of (SEQ ID NO:24).

22. A method of isolating a high mannose glycoform of a glycoprotein in a sample, wherein the sample comprises the high mannose glycoform and at least one other glycoform of the glycoprotein, the method comprising:
(a) loading the sample onto an ion exchange column under conditions that permit the retention of the glycoprotein onto the ion exchange column;
(b) passing an elution buffer through the ion exchange column to elute the glycoprotein from the ion exchange column;
(c) collecting a first one or more fractions that elute from the ion exchange column and contain the high mannose glycoform; and
(d) excluding from the first one or more fractions a second one or more fractions, wherein the second one or more fractions elute from the ion exchange column before or after the first one or more fractions and contain the at least one other glycoform, thereby isolating the high mannose glycoform in the sample.

23. The method of claim 22, wherein the ion exchange column is a cation exchange column and wherein the first one or more fractions that contain the high mannose glycoform elute from the cation exchange column after the second one or more fractions that contain the at least one other glycoform.

24. The method of claim 22, wherein the ion exchange column is an anion exchange column and wherein the first one or more fractions that contain the high mannose glycoform elute from the anion exchange column before the second one or more fractions that contain the at least one other glycoform.
